(19) United States
(12) Patent Application Publication
(10) Pub. No.: US 2020/0352795 A1
(43) Pub. Date: Nov. 12, 2020

(54) WETNESS INDICATOR WITH HARDENERS

(71) Applicant: SAVARE' I.C. S.r.l., Milano (IT)

(72) Inventors: Italo Corzani, Chieti (IT); Thomas James Klofta, Cincinnati, OH (US)

(21) Appl. No.: 16/965,050

(22) PCT Filed: Jan. 24, 2019
(86) PCT No.: PCT/IB2019/050600

§ 371 Date: Jul. 27, 2020

(30) Foreign Application Priority Data
Jan. 25, 2018 (IT) 102018000001900

(51) Int. Cl. A61F 13/42; A61L 15/24; A61L 15/56; A61L 15/34; A61L 15/48; A61L 15/58; A61L 15/22; G01N 21/81

(52) U.S. Cl. CPC A61F 13/42; A61L 15/24; A61L 15/56; A61L 15/34; A61L 15/48; A61L 2300/22; A61L 15/225; G01N 21/81; A61L 2300/21; A61L 2300/802; A61L 15/58

(57) ABSTRACT

A wetness indicator composition comprising at least one pH-indicator colorant, from about 0.001% to about 75% by weight of at least one color-stabilizer, and from 0.1% to 70% by weight of at least one hardening agent, and wherein the wetness indicator has a defined and properly selected hot to cold solidification rate, hardness and melt point.

### FIELD OF INVENTION

Disclosed are wetness indicator hot-melt formulations that comprise, besides at least one pH-indicator colorant, also hardeners and color-stabilizers.

### BACKGROUND OF THE INVENTION

Many disposable hygienic absorbent articles comprise a wetness indicator. Said wetness indicator compositions comprise a pH-indicator colorant adapted to change in appearance, i.e., appear, disappear, change color, etc., upon contact with water-containing body-liquids such as urine, runny bowel movements, menses, etc., in the absorbent article. However, current pH-based wetness indicators may be unreliable, having issues with a sufficient color stability of the composition i.e. against possible and highly undesirable premature triggering (pre-triggering) of color change during storage and before use, even in the absence of urine or any other body-liquids; moreover, they also show limits as to processability and the variety of beginning and final color options. Therefore, there is a continuing need for effective novel wetness/fluid indicator compositions that can provide a variety of color options and a continuing need for ways to improve the processability and, even more importantly, the stability of such wetness indicators within the absorbent articles.

### SUMMARY OF THE INVENTION

Herein are disclosed wetness indicator hot-melt compositions, comprising at least one pH-indicator colorant, from about 0.001% to about 75% by weight of at least one color-stabilizer, and from about 0.1% to about 70% by weight of at least one hardening agent and having a melt point temperature from about 58° C. to about 135° C., wherein the wetness indicator hot-melt composition has a hot to cold solidification rate of Delta(G′)/Delta(° C.) from about 3,800 to about 27,000 Pa/° C.;

### DETAILED DESCRIPTION OF THE INVENTION

### Definitions

“Absorbent article” or “hygienic article” refers to devices which absorb and contain physiological liquids. In some embodiments, absorbent article may refer to devices that absorb body exudates and, more specifically, may refer to devices which are placed against or in proximity to the body of the wearer to absorb and contain the various exudates or body fluids discharged from the body. Absorbent articles may include, but are not limited to, diapers, training pants, adult incontinence undergarments, feminine hygiene products, breast pads, bibs, and the like. As used herein, the terms “body fluids” or “body exudates” include, but are not limited to, urine, blood, vaginal discharges, breast milk, sweat and faecal matter.

“Comprise,” “comprising,” and “comprises” are open ended terms, each specifies the presence of what follows, e.g., a component, but does not preclude the presence of other features, e.g., elements, steps, components known in the art, or disclosed herein.

“Consisting essentially of” is used herein to limit the scope of subject matter, such as that in a claim, to the specified materials or steps and those that do not materially affect the basic and novel characteristics of the subject matter.

### DESCRIPTION OF THE INVENTION

An important consideration for a wetness indicator hot-melt composition is its processability, or its ability to easily and quickly be made and applied to an absorbent article. Ideally, a hot-melt wetness indicator sets up/solidifies, i.e. changes from a molten fluid into a solid, in a time quick enough to stay in the place it is placed, but not too fast, before it can spread and penetrate slightly into the material it is placed on. The wetness indicators hot-melt compositions of the present invention have a set point and a hot to cold solidification rate that allow high-temperature application to the article, which allows for better control.

Many wetness indicators comprise a colorant that is a pH indicator, i.e. a material that changes color when a pH change occurs. This mechanism of color change where the pH controls the hue of the color is called halochromism. This color change based on pH is most typically used for urine indicators employed in diapers. For such halochromic pH-indicator colorants, the negative logarithm of its acid dissociation constant, or pKa, can be a way to measure or predict at what pH the material's color will change when contacted by aqueous fluids like pure water or urine or other aqueous based solutions. Typically, most diaper wetness indicator compositions employ pH indicator colorants that possess pKa values that are acidic and below a value of 7. indicator

An example of a halochromic pH-indicator colorant is the free acid form of bromocresol green which has a pKa value around 4.6. Thus, for an aqueous solution of bromocresol green, if one were to maintain a solution pH below 4.6, over 50% of the bromocresol green molecules would be protonated and yellow in color. If one were to raise the bromocresol green solution pH above 4.6, over 50% of the molecules would be in their blue-green anionic and conjugate base state. If one were to maintain a pH of exactly 4.6, the color of this aqueous solution would be the result of combining 50% of the yellow molecules with 50% of the blue-green anionic molecules. For wetness indicator compositions possessing acidic pKa values below 7, like bromocresol green, the pH indicator colorant within the wetness indicator composition will be acidified in the dry state composition so it is maintained in its free acid form. For bromocresol green, its free acid form color is yellow, and this neutral free acid form is more easily formulated into polymeric wetness indicator hot-melt composition, such as the ones described in the present invention. Within the wetness indicator hot-melt composition containing bromocresol green, the neutral and protonated yellow acid form is stabilized by the addition of soluble acidic materials. Some pH indicator colorants possess a desirable yellow color when they are acidified to a pH below their pKa values. Some examples include bromocresol purple, bromocresol green and bromophenol blue which are all various shades of yellow in their free acid forms when they are acidified and protonated below their pKa values. Many of the acids used to maintain the free acid form of the pH indicator colorant in the polymeric wetness indicator hot-melt composition contain acid moieties like carboxylic acid groups or phosphate acid groups or sulfonic acid group and other acidic moieties. The acids most typically possess pKa values lower than the pH indicator colorants to keep them in their protonated form. As noted below, these acids are also called color-stabilizers.

Many of the current commercial wetness indicator compositions lack a sufficient stability against possible premature and undesirable color changes even in the dry state and in the absence of any liquid, for example due to storage in environmental conditions of high temperature and high relative humidity.

Therefore, the pH indicator colorants used in the wetness indicator hot-melt compositions of the present invention can have both an acidic or an alkaline/basic character. Therefore, they are, first of all, stabilized respectively in their free acid or free base dry form with the addition of controlled amounts of one or more properly selected color-stabilizers that, in the two cases, are acidic or basic color-stabilizers. In other words, if the pH indicator colorant is acidic, the function of the acid color-stabilizer is to maintain the desired dry state acidic color of the pH-indicator colorant within the wetness indicator composition until it is directly insulted with a higher pH body fluid like urine. Thus, a good performing color-stabilizer will even maintain the desired dry state acidic color of the pH-indicator colorant within the wetness indicator after the diaper or diapers within the package are stored at high temperature and air relative humidity. E.g., for pH indicator colorants with pKa values below 7, the color-stabilizer, being in this case an acid, helps to insure the pH indicator remains in its acidic and first color acidified dry state. This low pH color state of the pH-indicator colorant is formed because the opportune color-stabilizer is selected such to be more acidic and to have a lower pKa than the pH-indicator colorant. If the pKa of the color-stabilizer is lower than the pKa of the pH indicator colorant, the color-stabilizer is more acidic than the pH indicator colorant. The same is of course valid for basic color-stabilizer with basic pH-indicator colorants.

Both the lower pKa and opportunely relatively high concentration of the acid color-stabilizer versus the amount and acidity of the pH indicator colorant insures that the colorant stays in its acidic dry color state within the dry diaper until a color change is triggered by the higher pH of a massive quantity of urine (pH on average equal about 6), and/or other bodily exudates, which equally have a higher pKa than either the color-stabilizer or pH-indicator colorant. It is important to carefully optimize the amount of color-stabilizer in the wetness indicator hot-melt composition: as a too little amount may not stabilize the pH-indicator colorant against undesired premature colorant changes, due for example to too humid and hot climatic storage conditions or also to accidental contacts, within the stored diaper itself, with basic substances; while a too high amount of acid or basic color-stabilizer can negatively affect both the intensity of the color change and the kinetics of said color change or may even fully prevent it. Therefore, more in general. it is important to carefully optimize both the acidity or basicity of the color-stabilizer as characterized by its pKa, along with the concentration of said acidic or basic color-stabilizer. The rise in pH above or below the pKa's for both the color-stabilizer and pH-indicator colorant is the result of contact with the higher pH of the urine which, in case of acidic pH-indicator colorants and of the acidic color stabilizer, has a pKa higher than both the acid pH-indicator colorant and the acid color-stabilizer; similarly, for basic pH-indicator colorant, urine has a pH and a pKa lower than both the basic pH-indicator colorant and the basic color-stabilizer. Since the pKa of the urine is higher or lower than both the pH-indicator colorant and acid or basic color-stabilizer, the conjugate base or acid and anionic or cationic forms of pH indicator colorants and stabilizers can be formed. From now on, for simplicity we will mainly refer to acidic pH-indicator colorants therefore stabilized in their color by acidic color-stabilizers; but it's obvious that equal and parallel considerations are valid for basic pH-indicator colorants stabilized in their color by basic color-stabilizers.

In both cases, of pH-indicator colorants that have an acidic or an alkaline/basic character, the respective acid or basic color-stabilizers will be added, depending on their pKa and the pKa of the pH-indicator colorant, at a level sufficient to keep the colorant in a slightly acidic or alkaline/basic environment, just below or above the pH that cause its color change; therefore keeping and stabilizing the pH-indicator colorant in its primitive acidic or alkaline/basic color. A too low amount of color-stabilizer(s) would obviously render their action ineffective; while too high levels of color-stabilizer(s) would slow down the kinetics of color-change due to the change in pH caused by the contact with urine. Practically this means that color-stabilizers are present, in the hot-melt compositions of the present invention, in a quantity from about 0.001% to about 75% by weight of the composition, as a color-stabilizing system which may comprise one color-stabilizer or a combination/blend of color-stabilizers disclosed herein below; preferably from about 0.1% to about 70% by weight of the composition.

For example, for the acidic pH-indicator colorant bromocresol green, its anionic conjugate base form is blue-green in color. This conversion to the conjugate base form of the pH indicator colorant molecule results in a color change in use due to the higher pH of aqueous urine. Moreover, while some of the known wetness indicators may function sufficiently, the color options that are available in such systems are limited due to cost, formulation stability and processability, consumer color preferences, safety and purity constraints. Thus, there is a continuing need for wetness indicators with a variety of color options for both the first and second color states. Even though multiple color options are possible, it is in any case imperative that the dry state color of the wetness indicator is stable during various storage and shipping scenarios that can occur from the plant where the diaper is manufactured to the ultimate placement of the diaper on a baby. For example, the color of the wetness indicator must be stable after a consumer might store the diaper, or package of diapers, within a hot and humid environment. Moreover, and very important, the dry state color of the wetness indicator must be also stable to accidental contacts with other components within the diaper; especially those that are strongly alkaline and can migrate to contact and possibly pre-trigger the color change of the wetness indicator composition. Particularly dangerous and harmful, as a possible cause for this highly undesirable pre-triggering of the color change while still the diaper is not yet in use, is the possible contact, within a stored diaper, between the wetness indicator hot-melt composition and the granules of superabsorbent polymers. These superabsorbent polymers, widely used in the manufacturing of diapers and of other hygienic absorbent articles, in the dry state appear as small round granules that are very hard and that, being composed substantially by sodium polyacrylate, have a quite high pH, in the order of 8 or even more, capable to cause the change of color of a wetness indicator composition by simple accidental mechanical contact.

Interestingly, it has been found in the present invention that, by properly increasing the hardness of components within the disclosed wetness indicator hot-melt compositions can dramatically improve the stability in storage of the present wetness indicator hot-melt compositions also against color change pre-triggering due to the presence (and possible contacts), into hygienic absorbent articles, of strongly basic components like superabsorbent polymers. Thus, the additional presence of hardeners in opportune quantities, can contribute to an unexpectedly good improvement of the stability of the dry state color of the present wetness indicator hot-melt compositions, avoiding any premature color change before use on the baby/wearer. In particular, it has been found that opportune quantities of hardeners can make the present wetness indicator hot-melt compositions unexpectedly much more stable and more resistant to potential basic pre-triggers within the diaper.

As well known to all persons with an average expertise in the field of polymeric blends, and more in particular of hot-melt adhesives, harder components of a polymeric blend in most cases coincide with components that have a high degree of crystallinity, being, in a polymer, crystalline phases much harder than amorphous phases. Therefore, the hardeners preferably used in the present invention also contain a high level of crystallinity; and, this crystallinity, as again well known to the averagely expert person, may further induce, by a nucleating action, the rapid crystallization also of other polymeric components present in the hot-melt. For these reasons, the hardeners, used herein, that are already per se “hard” because they are “crystalline”, can further increase the global hardness of the whole hot-melt, by causing the crystallization also of other components, and by accelerating the speed at which this crystallization occurs. In other words, the crystalline hardeners preferably used in the present invention act also as “crystallizers” and “accelerators of crystallization” (and therefore of “hardening”) for the whole hot-melt, besides their own high hardness and crystallinity.

A very interesting consequence of this speeding-up by the hardeners of the crystallization and therefore of the solidification-speed of the present hot-melt compositions, when they are applied from the molten state, is the following: the high speed of solidification/crystallization from the melt of the present hardened hot-melt compositions, effectively inhibits the potential migration of the wetness indicator compositions, in a fluid or semi-fluid state, to regions where pre-triggers, e.g. alkaline substance, like superabsorbent granules, are positioned within the diaper, in this way contributing to prevent even better a highly undesirable premature color change even in the dry state, in the absence of urine, preventing even the physical contact between the wetness indicator hot-melts and such potential pre-triggers.

A “pre-trigger” is herein defined as any material or outside physical event that can cause the desired dry state color to change in color prematurely and in the absence of any aqueous liquid. For example, the physical property of high temperatures can act as a pre-trigger and cause the dry state color of poorly stabilized wetness indicators to change color due to oxidation. In some cases, high relative humidity of air, e.g. in summer, can act as a pre-trigger to cause the dry state color to prematurely change to its wet state color even when actually no aqueous liquid is present. Within a diaper itself, basic/alkaline materials like in particular the above mentioned super-absorbent polymers (called also absorbent gelling materials, but also fillers like TiO₂ and calcium carbonate, alkaline surfactants, film and nonwoven materials, and even some adhesives that can contact the wetness indicator hot-melts can also act as highly noxious pre-triggers capable of undesirably changing the dry state color of the wetness indicator even during the storage of the absorbent article, and when it is not yet in use/in contact with a body-fluid. For example, the pre-trigger could change the wetness indicator's dry state color of yellow to its wet state color of blue-green even before being contacted by a fluid like urine. Or a pre-trigger like high temperatures might oxidize the dry state yellow color to an undesirable dark orange color.

Not to be bound by theory, but it is hypothesized that a harder wetness indicator hot-melt composition can resist, without any change in its dry color, the contact and deformation by these other pre-triggering materials that have higher pKa's, i.e. are more alkaline, than the pH-indicator colorant. Today, many manufacturers of absorbent articles pack their diapers under high pressures to maximize the number of articles within the package in order to reduce material and shipping costs. Because of these high pressures inside packages, there is a more intimate contact between the diapers and all the materials within a given diaper. Materials that possess pKa values higher than both the acid color-stabilizer and pH-indicator colorant, like the small very hard granules of superabsorbent polymers, can be especially detrimental because their hard nature allows easier penetration into softer materials like wetness indicator hot-melt compositions. In addition, the much higher pKa/alkalinity of these hard granule pre-triggers convert the pH-indicator colorant of a pH indicator composition, by simple mechanical contact even more rapidly if under pressure inside the package, to its wet state color of its conjugate base even in the absence of any liquids. Thus, if the contact area and the penetration of hard granules of superabsorbent polymers into the wetness indicator hot-melt composition is hindered due to the higher hardness of said hot-melt composition itself, the dry state stability of the present wetness indicator (WI) hot-melt compositions is unexpectedly strongly enhanced. Thus, the best stability performance for wetness indicator hot-melt compositions with pH-indicator colorants with pKa values below 7—i.e. that are acidic—is observed when an opportunely hardened wetness indicator hot-melt composition is combined with the optimum concentration of an acid color-stabilizer which possesses a pKa lower than the pKa of the pH-indicator colorant, while also applying the wetness indicator composition under conditions where it solidifies in a sufficiently quick way upon the material to which it is applied, e.g. through the addition of suitable amounts of some crystalline hardeners.

The hardener or hardening agent may be defined as a material formulated within the wetness indicator hot-melt composition in order to reduce the deformation that might be caused by another material within the diaper where this material may or may not be under pressure or in environments at high temperature and high relative humidity. As defined in the 10^{th} edition of The Condensed Chemical Dictionary (as revised by Gessner G. Hawley), hardness is the resistance of a material to deformation of an indenter of specific size and shape under a known load. Thus, a harder wetness indicator hot-melt composition can resist deformation from more alkaline hard materials like the superabsorbent polymers' granules that, inside a diaper, are under pressure and within close proximity to the wetness indicator.

As noted, effective organic hardeners, disclosed in the present invention, are also effective crystallizers due to their highly crystalline nature that e.g. may derive from their linear molecular structure which can speed up the nucleation and ultimate solidification of the composition. For improved wetness indicator color stability, it is important for the wetness indicator hot-melt composition to harden and solidify as quickly as possible upon the substrate, so it has limited chance of migrating to other regions of the diaper where pre-triggers, and especially hard granules of superabsorbent polymers, are positioned and present. Thus, as already mentioned, it is optimum to use crystalline hardeners that therefore solidify quickly to prevent migration of the wetness indicator hot-melt composition as it cools upon the substrate immediately after its application from the molten state. Most typically, the wetness indicator hot-melt composition is melted into its molten liquid state to be applied as hot and molten liquid at high temperature. But, the various performance features of the wetness indicator are most effectively communicated to the consumer when they are in the solid state near or on the backsheet-film of the diaper. As already emphasized, one fundamental characteristic of these performance features is its color stability, where the consumer expects the wetness indicator to possess the correct and consistent dry state color in every phase of the diaper's life before use, along with the expected rapid and well evident color change after the baby urinates within the diaper. Caregivers also expect the color difference between the dry state and wet states in use to be suitably different and very easily visible, so it is easy to detect a wetness event within the diaper and change it as soon as possible.

As far as processing of the wetness indicator hot-melt composition, if the time period between its liquid molten state at the applicator and the solid state on the diaper is shortened, a more stable wetness indicator diaper is obtained. Not to be bound by theory, but the crystalline hardener can speed up the nucleation and hot to cold solidification rate since the linear and more ordered crystalline molecules can line up with one another more quickly to form a harder solid hot-melt composition within the diaper. Also, this properly accelerated speed of solidification contributes to strongly improving the stability of the wetness indicator within the diaper, by avoiding possible and deleterious migrations, during the application, of the hot and molten composition to regions of the diaper where possible pre-triggers are positioned and present.

A measurement technique used to simulate the application, from the molten state, into a diaper of the wetness indicator hot-melt composition and to measure this hot to cold solidification rate, employs a Rheometer to measure the Delta(G′)/Delta(° C.). This parameter is calculated by dividing DeltaG′ (the change in the Elastic Modulus G′ in units of Pascals) by Delta® C. (the change in temperature in units of degrees Celsius). This rheological measurement, that reproduces the industrial manufacturing process of a hygienic article to which a wetness indicator hot-melt composition is applied from the molten state, is called the hot to cold solidification rate since the wetness indicator is first heated well above its melt point and then slowly cooled down. In this rheological measurement, the Rheometer is set up to measure the storage modulus G′ of the composition as it is cooled down at a controlled slow rate, equal to 2° C./minute, from the molten state at the starting temperature of 145° C. to the final temperature in the solid state of 5° C. As the temperature of the composition is decreased, the storage modulus G′ will eventually increase precipitously when the composition becomes solid. This happens around the so called “crossover point” or “crossover temperature”, i.e. in the point and region of temperatures (above room temperature) where the two Moduli of the material cross (see below for more details) i.e. where the Elastic Modulus G′ and the Viscous Modulus G″ are equal. In Rheology this crossover point of the Elastic and Viscous Moduli forms the “rheological melt point/temperature” or inversely the “rheological solidification point” of the material (see later for a more detailed discussion). The slope of this sharply descending region of G′, around its crossover temperature, is termed the “hot to cold solidification rate.” By plotting the storage modulus G′ in units of Pascals (Pa) on the ordinate (Y-axis) and the temperature in Celsius units (° C.) on the abscissa (X-axis), one can calculate the slope by dividing the change in storage modulus, DeltaG′, by the change in temperature, Delta® C., and arrive at the cold to hot-melting rate ratio of Delta(G′)/Delta(° C.). The units of this hot to cold solidification rate expressed as Delta(G′)/Delta(° C.) are Pa/° C.

As noted, this value of Delta(G′)/Delta(° C.) of the present wetness indicator hot-melt compositions directly correlates with the same wetness indicator's solidification rate in the actual industrial manufacturing process and it is optimum for it to be high so it can solidify quickly to avoid migration on or into neighbouring materials of the diaper, with the danger of possibly contacting superabsorbent polymers' granules, that act as deleterious color pre-triggers.

It also worth noticing that, as already mentioned and as well-known to every person averagely expert in Rheology and rheological measurements, during the above described experiment for the measurement of the Elastic Modulus G′ as a function of temperature, the Rheometer automatically measures and records also other rheological parameters as a function of temperature, in particular the Viscous Modulus G″ and the Tan Delta. Therefore, in the same experiment described above, it is also possible to determine the “crossover temperature (or point)” i.e. that value of temperature, above room temperature, at which the two Moduli cross/have the same value or also (which is equivalent by definition) where Tan Delta is equal to 1. Said crossover temperature, as described in more details below, is also considered in Rheology the so cold “rheological melting temperature” or “melt point” of a certain material. In one embodiment, the hot to cold solidification rate Delta(G′)/Delta(° C.) may be from about 3,800 to about 27,000 Pa/° C. In some embodiments, the hot to cold solidification rate may be from about 4,800 to about 22,600 Pa/° C. In some other embodiments, the hot to cold solidification rate may be from about 5,800 to about 17,800 Pa/° C.

One example of a suitable type of crystalline hardeners is a paraffin wax which is made up of normal and saturated straight-chain or long-chain alkane hydrocarbons ranging in carbon lengths most typically from C₁₈H₃₈ to C₃₂H₆₆. Due to the linear structure of the saturated normal alkanes within paraffin waxes, the straight-chain molecules can pack in close proximity with one another due to the high van der Waals forces that exist between their long and linear carbon chains and generate in this way hard crystalline regions. Examples of paraffin waxes include The International Group's (Titusville, Pa.) IGI-1230A, IGI-1250A, and IGI-1260A. Shell Wax 200 and 400. Other effective hardeners with a linear chain-structure include linear polyethylene like the Performalene M waxes (M70 wax, M80 wax, and M90 wax) from Baker Hughes Inc., and their Performalene polyethylene waxes like Performalene 400 and Performalene 655. Among waxes working as excellent hardening agents, those containing also acidic groups, like oxidized or maleated waxes or waxes derived from montanic acid or waxes that are copolymers of ethylene and maleic anhydride and maleic esters, as well as similar other acidic waxes, can be conveniently used in the present formulations. Linear primary and fully saturated alcohols also function well as hardening agents and these include stearyl alcohol, behenyl alcohol and higher molecular weight primary alcohols with an INCI name of C20-40 alcohols and possessing the trade name of Performacol 350, Performacol 425 and Performacol 550 from Baker Hughes Inc. Other appropriate hardeners include linear primary carboxylic acids like palmitic acid, stearic acid, behenic acid, or the higher melting point linear primary carboxylic acids trademarked as Unicid from Baker Hughes Inc. and Accucid line of higher molecular weight and linear primary carboxylic acids from the International Group. These higher molecular weight linear primary carboxylic acids include e.g. Unicid 350, Unicid 425 and Unicid™ 550 from Baker Hughes Inc. Given their acidic nature combined with their high surface hardness, the above mentioned linear primary carboxylic acids may function, in the present invention, at the same time as color-stabilizers, as well as hardening agents. Among non-acidic hardeners, also aliphatic crystalline polyesters may be usefully employed in the described wetness indicator hot-melt compositions.

The hardness of the wetness indicator compositions of the present invention can be measured for example by the so called “Needle-Penetration” method, as described in ASTM D1321-04. The formulations of the present invention have a Needle Penetration no greater than about 40 dmm at 23° C. and preferably no greater than about 170 dmm at 55° C.

It is obvious that too low levels of hardening agent(s) are ineffective in hardening the hot-melt compositions of the present invention to a hardness sufficient to give a physical stabilization of the composition against the contact with e.g. hard pre-triggers (e.g. granules of superabsorbent polymers) that are present in a hygienic absorbent article, and whose different pH may trigger an undesired color-change, even in the dry state and in absence of urine.

However, also excessive levels of hardening agent(s) may cause undesirable problems: e.g. hardeners, as noted may accelerate the solidification speed of the composition from the molten state. This is a very helpful feature, if this kinetics of solidification is comprised in the limits that will be indicated below. In fact a composition that solidifies too quickly from the molten state, owing to its excessive level of hardeners, causes severe problems in the process: for example it may prematurely solidify, before reaching the substrate on which it must be coated, in case clogging the extrusion-head; or, to avoid such a problem, it needs to be applied at a too high melt-temperature, therefore possibly burning, perforating or deforming thermosensitive substrates, like plastic films. Moreover, too hard hot-melt compositions are also fragile, as well known by all persons with an average expertise in hot-melt adhesives. For this reason, a too hard wetness indicator hot-melt would risk of being mechanically broken and detaching by handling, even before the use, therefore making totally useless its application inside an absorbent hygienic article.

A wetness indicator hot-melt composition may therefore typically comprise from about 0.1% to about 70% by weight of a hardener (hardening agent), which may be one hardener, or a combination of hardeners disclosed herein. In some embodiments, the amount of hardener in the wetness indicator may be at least about 5%, at least about 10%, at least about 20%, at least about 25%, at least about 30%, at least about 40%, at least about 50%, or even at least about 60% by weight. In some embodiments, the hardener may be from about 1% to about 70%, from about 10% to about 60%, from about 20% to about 50% by weight of the wetness indicator hot-melt composition.

For the reasons outlined above, the inclusion of properly dosed selected hardeners leads to improved stability of the wetness indicator within the diaper. But some hardeners possess excessively high melting points which can lead to excessively long times for the wetness indicator composition to melt in the tank or even worse, cause melting or thermal damaging of the thermosensitive substrates (plastic films and nonwovens) onto which the hot-melt composition is coated. So, a fundamental characteristic of the wetness indicator hot-melt composition is also its melt point (or melting temperature) in units of degrees Celsius (° C.). First of all, a correct range of melt point is important for the right mixing of the wetness indicator composition during manufacturing thereof, to insure uniform homogeneity of the composition and avoid oxidation and degradation by oxygen. A processing temperature a bit higher than the melt point (i.e. about 10° C. higher) is also opportune during the application for properly setting up the melt tank and slot coater application equipment in order to achieve a sufficiently low viscosity, to coat a quality pattern within the diaper and maintain stability within the equipment. In addition, the melt point is indicative of how stable the wetness indicator composition is during transport and storage along with its stability within the diaper. If the melt point is too high, the composition could be oxidatively degraded during its preparation; or, during its processing and application, the too hot hot-melt composition could melt and damage the many thermosensitive materials present within a diaper, like plastic films and nonwovens; or it can take excessively long to first melt within its melt tank etc. Vice versa if the melt point of the hot-melt composition is too low, this composition could soften and migrate throughout the diaper if for example the consumer inadvertently stores the product at high temperatures. In some embodiments, the melt point temperature of the wetness indicator hot-melt compositions according to the present invention may be from about 58° C. to about 135° C., and in other embodiments from about 65° C. to about 120° C.

As already mentioned for the wetness indicator compositions according to the present invention, the melt point temperature (or melting temperature) is preferentially defined according to a rheological criterion. So, the melt point temperature is defined as the value of temperature, above room temperature, at which the Elastic Modulus G′ and the Viscous Modulus G″ of the composition cross, or also (which is equivalent by definition) the temperature at which Tan Delta is equal to 1. In fact, at temperatures below said point, the Elastic Modulus G′ (that expresses the “solid character” of the material) prevails over the Viscous Modulus (that on the contrary expresses the “fluidity” of the material) and therefore the material behaves like a solid.

On the contrary, at temperatures above said point the opposite is true: G″ prevails over G′ and therefore the material behaves like a fluid or a liquid. This crossover temperature is well known in the literature as the “rheological melt point” of a certain substance; as mentioned, it can be measured as the “crossover temperature of the Moduli”, during the already described rheological experiment for the measure of the Elastic Modulus G′ (as well as of the other rheological parameters Viscous Modulus G″ and Tan Delta) as a function of temperature.

The wetness indicator hot-melt compositions of the present invention can also include more than one pH-indicator colorant, wherein each pH-indicator colorant is stabilized in its first color dry state with its own opportune color-stabilizer. Because the first pH-indicator colorant and the first color-stabilizer may have a similar pKa and the second pH-indicator colorant and the second color-stabilizer may have a similar pKa, each pH-indicator colorant in the present wetness indicator compositions may be maintained (or stabilized in color) in its first color state until triggered to its second color state by the presence of urine or other aqueous body exudate. In some cases, pKa's of the pH-indicator colorants may be low and can be stabilized with very acidic color-stabilizers. In other cases, pKa's may be high (over 7) and the pH-indicator colorants can, on the contrary, be stabilized in color with basic/alkaline color-stabilizers with high pKa values above 7. In any case, the use of customized color-stabilizers in the present invention can allow for a much greater variety of pH indicator-colorants that can be utilized in wetness indicator hot-melt compositions. This use of various combinations of pH-indicator colorants and color-stabilizers results in a large variety of both dry state and wet state colors for the present wetness indicator compositions. With optimum formulation design and with multiple pH-indicator colorants and multiple color-stabilizers, one can even trigger different colors to appear at different times after a body fluid like urine contacts the wetness indicator composition. In addition, the selection of the correct color-stabilizer can lead to enhanced dry state color-stabilization of the desired dry state color. As noted, this chemical color-stabilization along with formulations that set up as hard compositions and solidify as quickly as possible on the absorbent article, during application, can lead to an unexpectedly improved dry state color-stabilization of said wetness indicator hot-melt compositions before the absorbent article, e.g. a diaper, is used.

Currently, many diaper wetness indicators transition from a yellow dry state to a blue-green color after urine contacts the wetness indicator (WI) composition. This is due to the common choice of bromocresol green as the pH indicator colorant in various WI compositions. Bromocresol green is commonly used because its yellow to blue-green color change is well liked by care givers and its pKa of 4.6 is optimum for use in WI compositions. Also, its yellow free acid form is readily soluble in most organic or polymeric lipophilic ingredients used in hot-melt compositions. This pKa of 4.6 for bromocresol green is ideal since the yellow free acid state of bromocresol green can be stabilized in the dry state by the use of low-cost chemicals functionalized with carboxylic acid groups since many molecules possessing carboxylic acid moieties possess pKa's similar or lower than the pKa of bromocresol green. Depending on the chemical structure of the particular carboxylic acid, one can expect its pKa to be in the range of 3 to 5 which is typically acidic enough to convert the bromocresol green pH-indicator colorant into its yellow free acid form. Even though carboxylic acid moieties are ideally suited for a pH-indicator colorant like bromocresol green, they may not be strong enough acids for other pH-indicator colorants with lower and more acidic pKa values than bromocresol green. Thus, the color-stabilizer's pKa must be close, or preferably lower than the pKa of the pH-indicator colorant in order to form the free acid colored state in the dry state within an absorbent article like a diaper. Preferably, when the pH-indicator colorant is acidic, the color-stabilizer is a stronger acid and possesses a lower pKa than the colorant in order to ensure that it is completely protonated in its free acid color state. In addition, bromocresol green's pKa of 4.6 is much lower than the average pH of urine such that when wetted with urine, it quickly and efficiently changes to its blue-green color state as the proton is released from the bromocresol green and the conversion into the blue-green conjugate base state takes place. Thus, because its pKa is between the pKa of many carboxylic acid containing molecules and the pH of urine, bromocresol green is an optimum pH-indicator colorant with attractive dry and wet state colors. Also, bromocresol green possesses an attractive color change of yellow in its acidic dry state to a blue-green color after it is converted to its conjugate base form after the more alkaline urine contacts the wetness indicator.

Some caregivers would prefer different or additional color choices within their wetness indicators. For example, a color change of orange in the dry state before the diaper is put on the baby and blue when the baby urinates within the diaper, or yellow in the dry state and purple in the wet state. Here, for this change of yellow to purple, bromocresol purple might be an ideal candidate with its known color change of yellow in its free acid form and purple when it is deprotonated to its conjugate base form. But, bromocresol purple has a higher pKa of 6.3 compared to the pKa of 4.6 for bromocresol green. So, although bromocresol purple's higher pKa allows it to be easily stabilized in its yellow dry state with chemicals functionalized with carboxylic acid moieties since they are much more acidic than the bromocresol purple, the bromocresol purple does not easily change to purple upon contact with urine since its pKa is higher than the average pH of baby's urine. This close proximity of the urine's pH to the pKa of the bromophenol purple results in slow kinetics for the color change of the bromocresol purple and it can take a very long time for it to fully develop a clearly visible dark purple color. Depending on the acidity of the wetness indicator composition, the bromocresol purple may remain protonated and never change to purple in its conjugate base form. To achieve the dark purple color of bromocresol purple, one would have to raise the pH one to two units above its pKa value of 6.3. This insures that the bromocresol purple is in its highly conjugated and purple conjugate base form. One might add an alkaline ingredient to the wetness indicator composition to increase the pH upon urine contact, but this typically degrades and negatively affects the dry state stability of the yellow acidic color. Prior to use on one's baby, the alkaline additive could leach out of the wetness indicator composition, especially in humid environments, to increase the pH and convert the free acid into the purple conjugate base form. As noted, this dry state stability is especially challenging in humid environments where the moisture might solubilize and increase the activity of the added alkaline ingredient. The increased solubility of the alkaline ingredient could raise the pH above the pKa of the bromocresol purple and pre-trigger its color change to purple in the dry state.

The present invention discloses that dry and wet state colors can be formulated if at least two pH-indicator colorants are combined into a single formulation. For example, a wetness indicator system may comprise a first and second pH-indicator colorant and also a first and second color-stabilizer, where the first pH-indicator colorant and the first stabilizer have similar pKa's and the second pH-indicator colorant and second color-stabilizer have similar pKa's. As noted, the color-stabilizers maintain the desired dry state color of the colors when the wetness indicator composition is subjected to severe environmental conditions like high humidity and temperature or even when pre-triggers are present within the diaper which could destabilize the wetness indicator. Finally, hard compositions that set up quickly during the application process also contribute in stabilizing the composition; especially if pre-triggers, like in particular hard granules of superabsorbent polymers, are present in the diaper and in close proximity to the wetness indicator. In some embodiments, the first color-stabilizer's pKa is from about two units below to about one unit above the pKa of the first pH-indicator colorant, and the second color-stabilizer's pKa is from about two units below to about one unit above the pKa of the second pH-indicator colorant. In some embodiments, the pKa of the pH-indicator colorant and color-stabilizer may be from about 1.5 to about 3.5, while the pKa of the second pH-indicator colorant and color-stabilizer may be from about 3.0 to about 5.0, in some embodiments from about 3.5 to about 5.5.

For example, the combination of two pH-indicator colorants such as phloxine B acid and the free acid of bromophenol blue can provide either a color change from yellow to purple or from orange-red to purple. This can be accomplished by careful selection of the color-stabilizers for each of the pH-indicator colorants. For the yellow to purple color change, a phosphorous based color-stabilizer, like alkyl phosphates e.g. cetyl phosphate, has a pKa low enough to acidify both the phloxine with its pKa near 2.9 and the bromophenol blue with its pKa near 4.0. It should be noted that alkyl phosphate color-stabilizers like cetyl phosphate, stearyl phosphate and cetearyl phosphate can be complex mixtures of multiple molecules. Thus, a cetyl phosphate from a given supplier may contain traces of phosphoric acid, monocetyl phosphate, dicetyl phosphate and tricetyl phosphate. This combination can still be effective in acidifying the pH-indicator colorant because some or all the trace materials may be more acidic than the pH-indicator colorant. For example, phosphoric acid has a very low pKa value and so a color-stabilizer containing traces of phosphoric acid can still be very effective in acidifying pH-indicator colorants within the wetness indicator matrix. Further, if the color-stabilizer is substantially one molecule, meaning at least about 90% one molecule, in some cases at least about 95% one molecule, or in some cases at least about 99% one molecule, the pKa of the color-stabilizer may be considered to be the pKa of the predominating molecule. Many acid and base color-stabilizers will be a mixture of multiple acid ingredients or a mixture of multiple base ingredients, and a key property for their proper functioning within the wetness indicator composition is to be either a stronger acid or a stronger base respectively than the pH-indicator colorant they are stabilizing in its color. At a pH below their pKa values, the phloxine is colorless and the bromophenol blue is yellow. If the pH is above their pKa values, the phloxine is red and the bromophenol blue is blue such that the mixture of red and blue results in a final purple color in the wet state. Thus, the resulting dry state color is yellow and the resulting wet state color after being insulted with urine is purple for this combination. But, only a low concentration of the phosphorous based color-stabilizer can be used since it is much more acidic than the bromophenol blue while being closer in acidity to the phloxine. If one includes too much of the cetyl phosphate acid stabilizer which may contain traces of phosphoric acid, the urine might not be able to completely solvate and deprotonate the color-stabilizer. In such a case, there could be enough remaining acidic protons to keep both the phloxine and bromophenol blue in their protonated acid states. Being a strong acid with a pKa lower than both the pKa's of the phloxine and bromophenol blue, this cetyl phosphate color-stabilizer can stabilize in their colors both the phloxine and bromophenol blue into their free acid states. As noted, if too much phosphorous based acid is used as a color-stabilizer, the yellow dry state is achieved but the color change to purple after wetting with urine is very slow and the color is faint. This is because the strongly acidic phosphorous based color-stabilizer hinders the rise in pH above the pKa of the bromophenol blue. Essentially, the system can be too acidic such that the formation of the conjugate bases of the pH-indicator colorants is hindered or takes too a long time period after contact with the body fluid. To achieve the purple wet conjugate base state color with acceptable kinetics, a low level of the phosphorous based color-stabilizer acid like Clariant's Cetyl Phosphate (trade name of Hostaphat CC-100) is incorporated along with a carboxylic acid based ingredient for acidification of the bromophenol blue. For a wetness indicator hot-melt composition containing both pH-indicator colorants of the free acid of phloxine and the free acid of bromophenol blue, an optimum amount of Hostaphat CC-100 acidic color-stabilizer is around 0.5 to 1.5% by weight. This is equivalent to around 0.05% to 0.15% of elemental phosphorus being contributed from the color-stabilizer. Not being too strong of an acid color-stabilizer but possessing a pKa around that of the pH-indicator colorant, the carboxylic acid can keep the bromophenol blue colorant acidified in its yellow dry state, but it does not hinder the quick color change to purple after wetting with urine for this particular combination of phloxine, bromophenol blue, and the two acidic color-stabilizers. The carboxylic acid based color-stabilizer is strong enough, as an acid, to maintain the yellow dry state but not so strong as to hinder a rise in pH well above the pKa of the bromophenol blue after contact with baby's urine. The addition of the carboxylic acid based color-stabilizer also aids in maintaining the yellow dry color if the caregiver exposes the diaper to high humidity and temperature. Finally, since it possesses a linear and fully saturated alkyl chain and hard, crystalline structure, the Hostaphat CC-100 can also function as a hardening agent although its effectiveness would be limited since low concentrations are typically used.

### EXAMPLES

### Example 1

Example 1 is a wetness indicator hot-melt composition that changes from a yellow dry state color to a bluish-green wet state color and it contains three color-stabilizers with Foral AX-E, Hostaphat CC-100 and Unicid 550, that in this case must have an acidic character. The very hard color-stabilizer Unicid 550 also functions as a hardening agent. Thus, Example 1 possesses both chemical color-stability due to the inclusion of acid color-stabilizers and physical stabilization due to the inclusion of hardening agents, which allow the WI composition to set up quickly into a hard solid on the substrate. Its quick solidification on the substrate during manufacturing prevents it from migrating into other regions of the diaper where pre-triggers might be present. Example 1 hardness, as a solid, also inhibits penetration of pre-triggers, like the very hard granules of superabsorbent polymers, into the WI hot-melt composition, while the acid color-stabilizers maintain the protonated dry state colors of the pH-indicator colorants even in hot and humid environments.

| Example 1 - | | | |
|---|---|---|---|
| Yellow to | WAV | | |
| Blue-Green | (%) | CAS No. | Function |
| Performathox 450 | 10.0 | 251553-55-6 | Non-ionic |
| ethoxylate* | | | Surfactant |
| Performathox 480 | 20.0 | 251553-55-6 | Non-ionic |
| ethoxylate* | | | Surfactant |
| Foral AX- | 10.0 | 9005-00-9 | Tackifying |
| E [symbol] | | | Agent/Color- |
| | | | Stabilizer |
| Unicid 550^{Ω} | 58.3 | 251554-90-2 | Hardener/ |
| | | &9002-88-4 | Color- |
| | | | Stabilizer |
| Hostaphat ™ | 0.2 | 3539-43-3 | Color- |
| CC-100^{>} | | | Stabilizer |
| Irganox 1010 ^{□} | 1.0 | 1709-70-2 | Anti-Oxidant |
| Bromocresol | 0.5 | 76-60-8 | pH-indicator |
| Green Free | | | Colorant |
| Acid [symbol] | | | |

*Performathox 450 and Performathox 480 as supplied by Baker-Hughes of Houston, TX. [symbol]
Foral AX-E as supplied by Eastman Chemicals of Kingsport, TN.
^{□} Irganox 1010 as supplied by BASF of Florham Park, NJ.
^{>}Hostaphat ™ CC-100 as supplied by Clariant Inc. of Charlotte, NC
^{Ω}Unicid 550 as supplied by Baker-Hughes of Houston, TX. [symbol]
Bromophenol Green free acid as supplied by TCI Chemicals of Portland, OR.

### Example 2

Example 2 is a subtle modification of Example 1 with the addition of an acidic ethylene-acrylic copolymer that functions as both an acidic color-stabilizer and as a polymeric base for the hot-melt matrix. Here in Example 2, the ethylene-acrylic acid polymer AC 5120 from Honeywell raises the viscosity for improved processing during application while still setting up quickly to create a hard formula that is stable to most pre-triggers within the diaper and that also resists color changes when exposed to high temperatures and highly humid air. Example 2 is a wetness indicator composition that changes from a yellow dry state color to a bluish-green wet state color and it contains four color-stabilizers with Foral AX-E, Hostaphat CC-100, AC5120 from Honeywell Inc., and Unicid 550, that in this case are acids. As noted, the color-stabilizer Unicid 550 also functions as a hardener. Thus, this Example 2 composition possesses both chemical color-stability due to the inclusion of color-stabilizers and physical stabilization due to the inclusion of hardeners which allow the wetness indicator (WI) composition to set up quickly into a hard solid on the substrate. Its quick solidification on the substrate during manufacturing prevents it from migrating into other regions of the diaper where pre-triggers might be present. Example 2 hardness, as a solid, also inhibits penetration of pre-triggers, like hard granules of superabsorbent polymer, into the WI, while the color-stabilizers maintain the protonated dry state colors of the pH-indicator colorants even in hot and humid environments. In the following table the Example 2 composition is shown, which exhibits not only chemical color-stabilization but also physical stabilization via the inclusion of hardeners.

| Example 2 - | | | |
|---|---|---|---|
| Yellow to | WAV | | |
| Blue-Green | (%) | CAS No. | Function |
| Performathox 450 | 10.0 | 251553-55-6 | Non-ionic |
| ethoxylate* | | | Surfactant |
| Performathox 480 | 20.0 | 251553-55-6 | Non-ionic |
| ethoxylate* | | | Surfactant |
| Foral AX- | 10.0 | 9005-00-9 | Tackifying |
| E [symbol] | | | Agent/Color- |
| | | | Stabilizer |
| Ethylene Acrylic | 20.0 | 9010-77-9 | Color- |
| Acid copolymer | | & | Stabilizer/ |
| AC-5120^{Ω} | | 79-10-7 | Binding Agent |
| Unicid 550^{Ω} | 38.3 | 251554-90-2 | Hardener/Color- |
| | | &9002-88-4 | Stabilizer |
| Hostaphat ™ | 0.2 | 3539-43-3 | Color-Stabilizer |
| CC-100^{>} | | | |
| Irganox 1010 ^{□} | 1.0 | 1709-70-2 | Anti-Oxidant |
| Bromocresol Green Free | 0.5 | 76-60-8 | pH-indicator |
| Acid [symbol] | | | Colorant |

*Performathox 450 and Performathox 480 as supplied by Baker-Hughes of Houston, TX. [symbol]
Foral AX-E as supplied by Eastman Chemicals of Kingsport, TN.
^{□} Irganox 1010 as supplied by BASF of Florham Park, NJ.
^{>}Hostaphat CC-100 as supplied by Clariant Inc. of Charlotte, NC.
^{Ω}Ethylene Acrylic Acid as supplied as AC-5120 by Honeywell Inc. of Morristown, NJ.
^{Ω}Unicid 550 as supplied by Baker-Hughes of Houston, TX. [symbol]
Bromophenol Green free acid as supplied by TCI Chemicals of Portland, OR.

### Example 3

Example 3 is a subtle modification of Example 2 with the use of Unicid 350 instead of Unicid 550. Here, Example 3 is another wetness indicator composition that changes from a yellow dry state color to a bluish-green wet state color and it contains four color-stabilizers with Foral AX-E, Hostaphat CC-100, AC-5120 from Honeywell Inc., and Unicid 350, that in this case are again acidic in nature. As noted, the color-stabilizer Unicid 350 also functions as a hardener. Thus, this Example 3 composition possesses both chemical color-stability due to the inclusion of acidic color-stabilizers and physical stabilization due to the inclusion of hardeners which allow the WI composition to set up quickly into a hard solid on the substrate. Its quick solidification on the substrate during manufacturing prevents it from migrating into other regions of the diaper where pre-triggers might be present. Example 3 hardness, as a solid, also inhibits penetration of pre-triggers into the WI, while the acid stabilizers maintain the protonated dry state colors of the pH-indicator colorants even in hot and humid environments. In the following table the Example 3 composition is shown, which exhibits not only chemical color-stabilization but also physical stabilization via the inclusion of hardeners.

| Example 3 - | | | |
|---|---|---|---|
| Yellow to | WAV | | |
| Blue-Green | (%) | CAS No. | Function |
| Performathox 450 | 10.0 | 251553-55-6 | Surfactant |
| ethoxylate* | | | |
| Performathox 480 | 20.0 | 251553-55-6 | Surfactant |
| ethoxylate* | | | |
| Foral AX- | 10.0 | 9005-00-9 | Tackifying |
| E [symbol] | | | Agent/Color- |
| | | | Stabilizer |
| Ethylene Acrylic | 25.5 | 9010-77-9 | Color- |
| Acid copolymer | | & | Stabilizer/ |
| AC-5120^{Ω} | | 79-10-7 | Binding Agent |
| Unicid 350^{Ω} | 31.8 | 251554-90-2 | Hardener/ |
| | | &9002-88-4 | Color-Stabilizer |
| Hostaphat ™ CC-100* | 0.2 | 3539-43-3 | Color-Stabilizer |
| Irganox 1010 ^{□} | 2.0 | 1709-70-2 | Anti-Oxidant |
| Bromocresol | 0.5 | 76-60-8 | pH-indicator |
| Green Free | | | Colorant |
| Acid [symbol] | | | |

*Performathox 450 and Performathox 480 as supplied by Baker-Hughes of Houston, TX. [symbol]
Foral AX-E as supplied by Eastman Chemicals of Kingsport, TN.
^{□} Irganox 1010 as supplied by BASF of Florham Park, NJ.
^{>}Hostaphat CC-100 as supplied by Clariant Inc. of Charlotte, NC.
^{Ω}Ethylene Acrylic Acid as supplied as AC-5120 by Honeywell Inc. of Morristown, NJ.
^{Ω}Unicid 550 as supplied by Baker-Hughes of Houston, TX. [symbol]
Bromophenol Green free acid as supplied by TCI Chemicals of Portland, OR.

### Example 4

Example 4 is another modification of Example 2 and Example 3 with the use of Unicid 425 along Unicid 550. Here, Example 4 is another wetness indicator composition that changes from a yellow dry state color to a bluish-green wet state color and it contains five color-stabilizers with Foral AX-E, Hostaphat CC-100, AC-5120 from Honeywell Inc., and both Unicid425 and Unicid550, that in this case have an acidic character. As noted, the stabilizers Unicid 425 and Unicid550 also functions as hardeners. Thus, this Example 4 composition possesses both chemical color-stability due to the inclusion of color-stabilizers, in this case acidic ones, and physical stabilization due to the inclusion of hardeners which allow the WI composition to set up quickly into a hard solid on the substrate. Its quick solidification on the substrate, during manufacturing, prevents it from migrating into other regions of the diaper where pre-triggers might be present. Example 4 hardness, as a solid, also inhibits penetration of pre-triggers into the WI, while the color-stabilizers maintain the protonated dry state colors of the pH-indicator colorants even in hot and humid environments. In the following table the Example 4 composition is shown, which exhibits not only chemical color-stabilization but also physical stabilization via the inclusion of hardeners.

| Example 4 - | | | |
|---|---|---|---|
| Yellow to | WAV | | |
| Blue-Green | (%) | CAS No. | Function |
| Performathox 450 | 10.0 | 251553-55-6 | Surfactant |
| ethoxylate* | | | |
| Performathox 480 | 20.0 | 251553-55-6 | Surfactant |
| ethoxylate* | | | |
| Foral AX- | 10.0 | 9005-00-9 | Tackifying Agent/ |
| E [symbol] | | | Color-Stabilizer |
| Ethylene Acrylic | 25.5 | 9010-77-9 | Color-Stabilizer/ |
| Acid copolymer | | & | Binding Agent |
| AC-5120^{Ω} | | 79-10-7 | |
| Unicid 425^{Ω} | 24.0 | 251554-90-2 | Hardener/ |
| | | &9002-88-4 | Color-Stabilizer |
| Unicid 550^{Ω} | 7.3 | 251554-90-2 | Hardener/ |
| | | &9002-88-4 | Color-Stabilizer |
| Hostaphat ™ | 0.2 | 3539-43-3 | Color-Stabilizer |
| CC-100^{>} | | | |
| Silicone Oil | 0.5 | 63148-62-9 | Plasticizer |
| 10 cSt | | | |
| Irganox 1010 ^{□} | 2.0 | 1709-70-2 | Anti-Oxidant |
| Bromocresol | 0.5 | 76-60-8 | pH-indicator |
| Green Free | | | Colorant |
| Acid [symbol] | | | |

*Performathox 450 and Performathox 480 as supplied by Baker-Hughes of Houston, TX. [symbol]
Foral AX-E as supplied by Eastman Chemicals of Kingsport, TN.
^{□} Irganox 1010 as supplied by BASF of Florham Park, NJ.
^{>}Hostaphat CC-100 as supplied by Clariant Inc. of Charlotte, NC.
^{Ω}Ethylene Acrylic Acid as supplied as AC-5120 by Honeywell Inc. of Morristown, NJ.
^{Ω}Unicid 550 and 425 as supplied by Baker-Hughes of Houston, TX. [symbol]
Bromophenol Green free acid as supplied by TCI Chemicals of Portland, OR. Silicone Oil at 10 cSt as supplied by Dow Coming, Midland, MI

### Example 5

Example 5 shows a wetness indicator hot-melt composition with a yellow dry state that changes to purple upon contact with baby's urine. For this Example 5, there are multiple color-stabilizers where the main color-stabilizer for the free acid of bromophenol blue is the ethylene acrylic acid copolymer. The free acid of cetyl phosphate from the Hostaphat CC-100 is a strong enough acid to protonate both the Phloxine B acid into its colorless form and the bromophenol blue into its acidic yellow form. The hydrogenated acidic rosin tackifier trademarked as Foral AX-E from Eastman Chemicals can also function as both a tackifying agent along with functioning as a color-stabilizer. Being hydrogenated, the Foral AX-E is also of low color and low odor and is more stable than non-hydrogenated versions. The Hostaphat CC-100 cetyl phosphate stabilizer is acidic enough to protonate both the Phloxine B free acid and the free acid of bromophenol blue since the pKa of cetyl phosphate is lower than both of the pH-indicator colorants.

| Example 5 - | | | |
|---|---|---|---|
| Yellow to | WAV | | |
| Purple | (%) | CAS No. | Function |
| Performathox | 11.2 | 251553-55-6 | Non-ionic |
| 450 ethoxylate * | | | Surfactant |
| Performathox | 16.7 | 251553-55-6 | Non-ionic |
| 480 ethoxylate * | | | Surfactant |
| Foral AX- | 20.5 | 9005-00-9 | Tackifying |
| E [symbol] | | | Agent/Color- |
| | | | Stabilizer |
| Irganox 1010 ^{□} | 1.0 | 1709-70-2 | Anti-Oxidant |
| AC-5120 Ethylene | 40.0 | 9010-77-9 & | Color- |
| Acrylic Acid | | 79-10-7 | Stabilizer/ |
| copolymer^{Ω} | | | Binding Agent |
| Unicid 550^{Ω} | 5.0 | 251554-90-2 &9002- | Hardener/ |
| | | 88-4 | Color- |
| | | | Stabilizer |
| Benzoflex | 3.6 | 20109-39-1 | Plasticizer |
| 98-8^{‡} | | | |
| Hostaphat | 0.8 | 3539-43-3 | Color- |
| CC-100^{>} | | | Stabilizer |
| Tinuvin UV | 0.99 | 129757-67-1 & | UV Light |
| Light Protectants ^{∘} | | 127519-17-9 | Protectants |
| Bromophenol | 0.15 | 115-39-9 | pH-indicator |
| Blue Free | | | Colorant |
| Acid [symbol] | | | |
| Phloxine | 0.06 | 18472-87-2 | pH-indicator |
| B Acid♥ | | | Colorant |

* Performathox 420 and Performathox 480 as supplied by Baker-Hughes of Houston, TX. [symbol]
Foral AX-E as supplied by Eastman Chemicals of Kingsport, TN.
^{□} Irganox 1010 as supplied by BASF of Florham Park, NJ.
^{Ω}Ethylene Acrylic Acid as supplied as AC-5120 by Honeywell Inc. of Morristown, NJ.
^{‡}Benzoflex 98-8 as supplied by Eastman Chemicals of Kingsport, TN.
^{>}Hostaphat CC-100 as supplied by Clariant Inc. of Charlotte, NC.
^{∘} Tinuvin UV light protectants are a 50%-50% blend of Tinuvin 123 and Tinuvin 384-2 as supplied by BASF of Florham Park, NJ. [symbol]
Bromophenol Blue free acid as supplied by TCI Chemicals of Portland, OR.
♥Phloxine B Acid as supplied by TCI Chemicals of Portland, OR.
^{Ω}Unicid 550 as supplied by Baker-Hughes of Houston, TX.

pH-indicator colorants that may be used in the present invention include, but are not limited to, the pH-indicator colorants listed in Table 1 below. Table 1 also indicates the low pH color, the pH transition range, high pH color, and pKa of each pH-indicator colorant. (Orndorff, W. R.; Purdy, A. C. J. Am. Chem. Soc. 1926, 48, 2216; also in the book “The Sigma Aldrich Handbook of Stains, Dyes, and Indicators,” by Floyd J. Green, 2^{nd} printing published in 1991 by the Aldrich Chemical Company of Milwaukee, Wis.,; See, also, “The Handbook of Acid-Base Indicators,” by R. W. Sabnis and published in 2008 by CRC Press of NY, NY).

TABLE 1

| pH-INDICATOR | | Low pH | pH Transition | High pH | |
|---|---|---|---|---|---|
| COLORANT | CAS # | Color | Range | Color | pKa |
| Gentian Violet (Crystal | 548-62-9 | Yellow | 0.0 to 2.0 | Blue-Violet | 1.1 & 1.8 |
| Violet) | | | | | |
| Acid Phloxine B (free acid | 13473-26-2 | Colorless | 1.1 to 3.3 | Purple | 2.9 |
| form; D&C Red 27) | | | | | |
| Phloxine B (sodium salt; | 18472-87-2 | Colorless | 1.1 to 3.3 | Purple | 2.9 |
| D&C Red 28) | | | | | |
| Methyl Violet | 52080-58-7 | Yellow | 0.2 to 1.8 | Purple | 0.8 |
| Malachite Green (Acidic | 2437-29-8 | Yellow | 0.0 to 2.0 | Green | 1.3 |
| pH range) | | | | | |
| Malachite Green (Alkaline | 2437-29-8 | Blue-green | 11.6 to 14.0 | Colorless | 12.8 |
| pH range) | | | | | |
| Bromophenol Blue Free Acid | 115-39-9 | Yellow | 3.0 to 4.6 | Blue | 4.0 |
| Methyl Orange | 547-58-0 | Red | 3.2 to 4.4 | Yellow | 3.4 |
| Resazurin | 550-82-3 | Orange | 3.8 to 6.5 | Purple | 5.1 |
| Ethyl Red | 76058-33-8 | Red | 4.5 to 6.5 | Yellow | 5.4 |
| Bromocresol Green Free Acid | 76-60-8 | Yellow | 3.8 to 5.4 | Blue-Green | 4.8 |
| Quinaldine Red | 117-92-0 | Colorless | 1.4 to 3.2 | Red | 2.6 |
| Bromocresol Purple Free Acid | 115-40-2 | Yellow | 5.2 to 6.8 | Purple | 6.3 |
| Thymolphthalein | 125-20-2 | Colorless | 9.3 to 10.5 | Blue | 9.8 |
| Acid Fuchsin | 3244-88-0 | Red | 12.0 to 14.0 | Colorless | 13 |
| Nile Blue | 2381-85-3 | Blue | 9.4 to 11.0 | Purple-Red | 9.7 |
| Aniline Blue (also known | 28983-56-4 | Blue | 9.4 to 14.0 | Orange | 11.7 |
| as Methyl Blue) | | | | | |
| Indigo Carmine | 860-22-0 | Blue | 11.5 to 14.0 | Yellow | 12.7 |

The wetness indicator hot-melt compositions of the present invention may comprise from about 0.01% to about 15.0% by weight of pH-indicator colorant(s).

The wetness indicator hot-melt compositions may comprise additional standard permanent colorant(s), i.e. whose colors does not vary with pH, besides the pH-indicator colorant(s) discussed above. Additional suitable fluid colorants include water soluble colorants like direct dyes, acid dyes, base dyes, and various solvent-soluble colorants. Examples of colorants further include, but are not limited to, organic dyes, inorganic pigments, colored macromolecules, colored nanoparticles and materials. Some examples of oil soluble permanent colorants include D&C Yellow No. 11, D&C Red No. 17, D&C Red No. 21, D&C Red No. 27, D&C Red No. 31, D&C Violet No. 2, D&C Green No. 6, FD&C Red 3, D&C Orange No. 4, D&C Orange No. 17, and D&C Orange No. 5. Additional permanent colorants include Pigment Red 146 (CAS #5280-68-2), Pigment Red 122 (CAS #980-26-7), Pigment Orange 16 (CAS #6505-28-8), red beet extract, Manganese Phthalocyanine and other metallized phthalocyanines like copper phthalocyanines and metallized and alkylated porphyrin or phthalocyanines, and beta-carotene and mixtures thereof. Further appropriate additional colorants may include those listed in U.S. Ser. No. 62/147,258.

Appropriate color-stabilizers include, but are not limited to, those listed in the following Table 2, along with their pKa value(s). Some embodiments may use two, three, four, or more color-stabilizers. As noted above, the function of color-stabilizers, in case the pH-indicator colorant is an acid, is to keep the pH indicator colorant in a protonated state below its pKa value in the dry wetness indicator state. Similarly, alkaline color-stabilizers may also be required and here the function of the alkaline or basic color-stabilizer is to keep the pH indicator colorant in its conjugated basic form above its pKa value in the dry wetness indicator state. Thus, since pH indicator colorants have a plurality of different pKa's, a variety of different acids with varying pKa values are required to stabilize in color these various pH indicator colorants although in certain instances, one very strong acidic or basic color-stabilizer may perform very well with a variety of pH-indicator colorants. For the table of acidic and alkaline color-stabilizers below, some of them have more than one pKa value because that particular molecule has more than one acid or alkaline moiety. For example, citric acid possesses three acidic protons each of which having a different acid strength. Most frequently, the first pKa is the lowest since the first proton is most frequently the most acidic. Upon release of the first proton, the molecule becomes anionic and that negative charge makes it more difficult for the citric acid molecule to release the second proton. Thus, the second proton is less acidic than the release of the first proton and the second pKa (2) is higher than the first pKa (1). Finally, upon release of two protons from citric acid, the molecule now possesses a negative II charge and this attracts the last remaining positively charged proton such that it is the weakest proton of the three on the citric acid molecule. Thus, citric acid pKa (3) is larger than its pKa (2) for its second of three protons which is larger than the most acidic pKa (1) proton. In addition, some acid and alkaline color-stabilizers may be complex mixtures containing molecules with various pKa values. For example, and as noted, the cetyl phosphate acid color-stabilizer, sold as Hostaphat CC-100 from Clariant Inc., can contain traces of phosphoric acid and other acidic components. The key is that the acid or basic color-stabilizer has one or more components that can stabilize in color the pH-indicator colorant in its dry state. For an acid color-stabilizer, it must be more acidic and possess a lower pKa than the pH-indicator colorant it must acidify. For a basic color-stabilizer, it must be more alkaline and possess a higher pKa than the pH-indicator colorant, so that the alkaline colorant is maintained in its basic form in the dry state of the wetness indicator hot-melt composition.

TABLE 2

| COLOR STABILIZER | pKa | pKa | pKa | pKa | pKa |
|---|---|---|---|---|---|
| NAME | (1) | (2) | (3) | (4) | (5) |
| Acetamide | 0.6 | | | | |
| Acetic acid | 4.8 | | | | |
| Acetoacetic acid | 3.6 | | | | |
| Adipic acid | 4.4 | 5.4 | | | |
| Alkyl Sulfonic Acids | ~1 | | | | |
| 2-Aminobenzoic acid | 2.1 | 4.9 | | | |
| Ammonia | 9.2 | | | | |
| Aniline | 4.6 | | | | |
| Arginine | 1.8 | 9.0 | 12.5 | | |
| Ascorbic acid | 4.1 | 11.8 | | | |
| Aspartic acid | 2.0 | 3.9 | 10.0 | | |
| Barbituric acid | 4.0 | | | | |
| Benzenesulfonic acid | 0.7 | | | | |
| Benzoic acid | 4.2 | | | | |
| Benzylamine | 9.3 | | | | |
| Betaine | 1.83 | | | | |
| Boric acid | 9.3 | 12.7 | 13.8 | | |
| Butanoic acid | 4.8 | | | | |
| Butylamine | 10.8 | | | | |
| Carbonic acid | 6.4 | 10.3 | | | |
| Catechol | 9.4 | 12.8 | | | |
| Cetyl Phosphate | ~2 | | | | |
| Chloroacetic acid | 2.9 | | | | |
| Citric acid | 3.1 | 4.8 | 6.4 | | |
| m-Cresol | 10.0 | | | | |
| Cysteine | 1.7 | 8.4 | 10.8 | | |
| Decylamine | 10.6 | | | | |
| Dichloroacetic acid | 1.3 | | | | |
| Diethylamine | 10.9 | | | | |
| Diisopropylamine | 11.0 | | | | |
| Dimethylamine | 10.8 | | | | |
| Dimethylglyoxime | 10.7 | 12.0 | | | |
| Dinicotinic acid | 2.8 | | | | |
| Ethanolamine | 9.5 | | | | |
| Ethylamine | 10.6 | | | | |
| Ethylenediamine | 6.8 | 9.9 | | | |
| Ethylenediaminetetraacetic | −0.21 | 1.5 | 2.2 | 3.1 | 6.7 |
| acid (EDTA) | | | | | |
| Ethyleneimine | 8.0 | | | | |
| Formic acid | 3.7 | | | | |
| Fumaric acid | 3.1 | 4.5 | | | |
| L-Glutamic acid | 2.2 | 4.4 | 9.9 | | |
| L-Glutamine | 2.2 | 9.1 | | | |
| L-Glutathione | 2.12 | 3.59 | 8.75 | 9.65 | |
| Glyceric acid | 3.5 | | | | |
| Glycine | 2.3 | 9.8 | | | |
| Glycolic acid | 3.8 | | | | |
| Glyoxylic acid | 3.2 | | | | |
| Heptanedioic acid | 4.7 | | | | |
| Heptanoic acid | 4.9 | | | | |
| Heptylamine | 10.7 | | | | |
| Hexamethylenediamine | 11.9 | 10.8 | | | |
| Hexanoic acid | 4.8 | | | | |
| Hexylamine | 10.6 | | | | |
| Hydrogen chloride | −7 | | | | |
| Hydroquinone | 10.3 | | | | |
| Hydroxylamine | 5.9 | | | | |
| Lactic acid | 3.9 | | | | |
| Maleic acid | 1.9 | 6.3 | | | |
| Malic acid | 3.5 | 5.1 | | | |
| Malonic acid | 2.847 | 5.696 | | | |
| 4-Methylpentanoic acid | 4.8 | | | | |
| Nicotine | 3.1 | 8.0 | | | |
| Nitrous acid | 3.1 | | | | |
| Octadecylamine | 10.6 | | | | |
| Octanedioic acid | 4.5 | | | | |
| Octanoic acid | 4.9 | | | | |
| Oxalic acid | 1.2 | 4.3 | | | |
| Pentanoic acid | 4.8 | | | | |
| Perchloric acid | −10 | | | | |
| p-Periodic acid | 1.5 | 8.3 | | | |
| 1,10-Phenanthroline | 4.8 | | | | |
| Phenol | 10.0 | | | | |
| Phenylacetic acid | 4.3 | | | | |
| Phenylalanine | 2.2 | 9.3 | | | |
| Phenylethylamine | 9.8 | | | | |
| Phenylglycine | 1.8 | 4.4 | | | |
| Phosphoric acid | 2.1 | 7.2 | 12.4 | | |
| m-Phthalic acid | 3.5 | 4.6 | | | |
| o-Phthalic acid | 2.9 | 5.4 | | | |
| p-Phthalic acid | 3.5 | 4.8 | | | |
| Picolinic acid | 1.1 | 5.2 | | | |
| Picric acid | 0.4 | | | | |
| Propanoic acid | 4.9 | | | | |
| Propylamine | 10.6 | | | | |
| 3-Pyridinecarboxylic | 4.9 | | | | |
| acid | | | | | |
| 4-Pyridinecarboxylic | 5.0 | | | | |
| acid | | | | | |
| Pyrimidine | 6.3 | | | | |
| Pyrocatechol | 9.4 | 12.8 | | | |
| Pyrophosphoric Acid | 1.5 | 2.4 | 6.6 | 9.2 | |
| Pyrrolidine | 11.3 | | | | |
| Pyruvic acid | 2.4 | | | | |
| Quinine | 4.1 | 8.5 | | | |
| Quinoline | 4.9 | | | | |
| Resorcinol | 9.3 | 11.1 | | | |
| Salicylic acid | 3.0 | 13.7 | | | |
| Selenic acid | 1.9 | | | | |
| Selenous acid | 2.6 | 8.3 | | | |
| Serine | 2.2 | 9.0 | | | |
| o-Silicic acid | 9.7 | 11.7 | | | |
| m-Silicic acid | 9.7 | 12 | | | |
| Succinic acid | 4.2 | 5.6 | | | |
| Sulfuric acid | −3 | 2.0 | | | |
| Sulfurous acid | 1.9 | 7.2 | | | |
| d-Tartaric acid | 3.0 | 4.4 | | | |
| meso-Tartaric acid | 3.2 | 4.8 | | | |
| Terephthalic acid | 3.5 | | | | |
| m-Toluic acid | 4.3 | | | | |
| o-Toluic acid | 3.9 | | | | |
| p-Toluic acid | 4.4 | | | | |
| Trichloroacetic acid | 0.9 | | | | |
| Triethanolamine | 7.8 | | | | |
| Triethylamine | 10.7 | | | | |
| Trimethylacetic acid | 5.0 | | | | |
| Trimethylamine | 9.8 | | | | |
| Tris(hydroxymethyl)- | 8.1 | | | | |
| aminomethane (tris) | | | | | |
| Tyramine | 9.8 | 10.5 | | | |
| Tyrosine | 2.2 | 9.2 | 10.5 | | |
| Uric acid | 3.9 | | | | |

Table 3 below shows the hot to cold solidification rate and the melt point temperature for the five wetness indicator hot-melt compositions illustrated in the previous Examples.

TABLE 3

| | Hot to Cold | | |
|---|---|---|---|
| | Solidification rate - | | |
| | Delta G/Delta° C. | Melt | Met |
| Sample | (Pa/° C.) | point ° C. | Criteria? |
| WI Formula | 22618.86 | 113.82 | Yes |
| Example 1 | | | |
| WI Formula | 17819.8 | 102.75 | Yes |
| Example 2 | | | |
| WI Formula | 5779 | 81.7 | Yes |
| Example 3 | | | |
| WI Formula | 13564.06 | 100.34 | Yes |
| Example 4 | | | |
| WI Formula | 4817.94 | 73.5 | Yes |
| Example 5 | | | |

Table 4 shows the needle penetration at two temperatures for Examples 1-5 of the inventive wetness indicator hot-melt compositions. The low needle penetrations (measured according to ASTM D1321-04 in dmm or decimillimeters) indicate the high level of hardness for the disclosed wetness indicators, which leads to an unexpectedly improved color-stability in their dry state.

TABLE 4

| | Needle Penetration | Needle Penetration |
|---|---|---|
| | at 23° C. | at 55° C. |
| | (dmm) | (dmm) |
| WI Formula Example 1 | 0 | 32 |
| WI Formula Example 2 | 2 | 43 |
| WI Formula Example 3 | 5 | 102 |
| WI Formula Example 4 | 2 | 66 |
| WI Formula Example 5 | 7 | 115 |

Table 5 shows that the hardened hot-melt composition of the above Examples shows a superior resistance to pre-triggering (color change) for mechanical contact, even under pressure, with typical potential pre-triggers present in a diaper, like hard granules of superabsorbent polymers. The test was performed in the following way: a 35 g/m² coating of each formula under test, was coated from the melt on a polyethylene film. Then samples were cut as squares of about 5×5 cm (area=25 cm²). On the upwards sample was positioned a 10 g/m² polypropylene non-woven; and on the non-woven was sprinkled a layer of granules of a superabsorbent acrylic polymer Aqualic AC supplied by Nippon Shokubai (Japan). The material is supplied in small, irregularly sized spherical granules, with an average diameter between about 0.5 and 1 mm; rare larger granules have diameter between about 1 and 2 mm. The granules were sprinkled in a quantity of about 1 g/cm² so to obtain a homogeneously covering layer. This structure very well simulates the structure inside a baby absorbing-diaper, of how the various materials, like the wetness-indicator hot-melt composition and the granules of hard superabsorbent polymers, are mutually positioned.

A series of samples was tested without any pressure on; a second series of samples was tested by previously positioning a load on the whole above described structure so to obtain a pressure on the WI composition and on the layer of superabsorbent polymer equal to about 1.2 psi, equal to about 0.827 N/cm², that is the average pressure by which a diaper is typically squeezed inside a standard plastic bag for diapers.

A series of samples was tested at room conditions, i.e. 23° C. and 50% relative humidity; while another series of sample was tested in much more severe conditions for the color stability of the wetness indicator hot-melt compositions, i.e. at 40° C. and 75% relative humidity, to simulate very hot and humid tropical climates.

The test was considered passed if the samples retained their dry yellow color without any trace of overall color change (from yellow to blue-green for Examples 1 to 4, and from yellow to purple for Example 5) or of colored dots for at least 24 hours and preferably up to 72 hours (3 days); or if, when showing very rare microscopic colored dots even in the most severe conditions (under pressure, at 40° C. and 75% relative humidity at 3 to 7 days), the samples showed no more than 5 tiny colored micro-dots (diameter about 0.1-0.2 mm), per every 25 cm² of area of the samples, micro-dots due to the contact with some particularly large granules of the pre-triggering superabsorbent polymer.

TABLE 5

| | WI Formula | WI Formula | WI Formula | WI Formula | WI Formula |
|---|---|---|---|---|---|
| | Example 1 | Example 2 | Example 3 | Example 4 | Example 5 |
| 24 hours - room | YELLOW | YELLOW | YELLOW | YELLOW | YELLOW |
| conditions - no | No overall | No overall | No overall | No overall | No overall |
| pressure | color-change | color-change | color-change | color-change | color-change |
| | of the sample | of the sample | of the sample | of the sample | of the sample |
| | area/No | area/No | area/No | area/No | area/No |
| | colored dots | colored dots | colored dots | colored dots | colored dots |
| 72 hours - room | AS ABOVE | AS ABOVE | AS ABOVE | AS ABOVE | AS ABOVE |
| conditions - no | | | | | |
| pressure | | | | | |
| 7 days - room | AS ABOVE | AS ABOVE | AS ABOVE | AS ABOVE | AS ABOVE |
| conditions - no | | | | | |
| pressure | | | | | |
| 24 hours - room | AS ABOVE | AS ABOVE | AS ABOVE | AS ABOVE | AS ABOVE |
| conditions - under | | | | | |
| pressure 1.2 psi | | | | | |
| 72 hours - room | AS ABOVE | AS ABOVE | AS ABOVE | AS ABOVE | AS ABOVE |
| conditions - under | | | | | |
| pressure 1.2 psi | | | | | |
| 7 days - room | AS ABOVE | AS ABOVE | AS ABOVE | AS ABOVE | AS ABOVE |
| conditions - under | | | | | |
| pressure 1.2 psi | | | | | |
| 24 hours - 40° C./ | AS ABOVE | AS ABOVE | AS ABOVE | AS ABOVE | AS ABOVE |
| 75% R.H. - no | | | | | |
| pressure | | | | | |
| 72 hours - | AS ABOVE | AS ABOVE | AS ABOVE | AS ABOVE | AS ABOVE |
| 40° C./75% R.H. - no | | | | | |
| pressure | | | | | |
| 7 days - 40° C./ | AS ABOVE | AS ABOVE | AS ABOVE | AS ABOVE | AS ABOVE |
| 75% R.H. - no pressure | | | | | |
| 24 hours - 40° C./ | AS ABOVE | AS ABOVE | AS ABOVE | AS ABOVE | AS ABOVE |
| 75% R.H. - under | | | | | |
| pressure 1.2 psi | | | | | |
| 72 hours - 40° C./ | AS ABOVE | AS ABOVE | AS ABOVE | AS ABOVE | No overall color- |
| 75% R.H. - under | | | | | change of the sample |
| pressure 1.2 psi | | | | | area from Yellow - |
| | | | | | 1 purple micro-dot |
| | | | | | (≤0.1 mm) |
| 7 days - 40° C./ | AS ABOVE | AS ABOVE | No overall color- | AS ABOVE | No overall color- |
| 75% R.H. - under | | | change of the sample | | change of the sample |
| pressure 1.2 psi | | | area from Yellow - | | area from Yellow- |
| | | | 2 bluish- green | | 4 purple micro-dots |
| | | | micro-dots | | (≤0.1 mm) |
| | | | (≤0.1 mm) | | |

### Hot Melt Binding Matrix

The wetness indicator compositions that are utilized in this invention comprise a hot melt-binding matrix. Processing a hot-melt binding matrix involves melting the components together at a high temperature, typically from at least about 50° C. to about 170° C., in some embodiments, from about 60° C. to about 130° C., in some embodiments from about 80° C. to about 120° C. In order to be hot-melt processable, the wetness indicator composition must be heated to a temperature high enough (e.g. about 10° C. above its melting point) to insure the adhesive flows readily but not so hot to cause degradation at an unacceptable rate. Thus, it is common to add an anti-oxidant to hot-melt compositions in order to slow down the decomposition rate.

The hot-melt binding matrix may comprise first of all one or more thermoplastic polymer. A number of different polymers and blends of polymers may be used in the hot-melt compositions of the present invention as the primary binding agent(s) to combine and mix the pH indicator colorants with the acid or alkaline color-stabilizer, with the hardener(s), as well as with other optional ingredients such as tackifiers, waxes, surfactants, viscosity modifiers, fillers, anti-oxidants, UV stabilizers and other permanent colorants. As already said, some of these materials can have the ability to perform multiple contemporary functions; that is, they can function at the same time as hardeners or binding agents to contribute to the color-stability of the wetness indicator composition.

Such hot-melt thermoplastic polymers, copolymers, terpolymers, and other materials that can function as a primary binding agent include ethylene vinyl acetates (EVA), polyolefins like low density polyethylene (LDPE) and high density polyethylene (HDPE), amorphous polyolefins like atactic polypropylene and polypropylene homopolymers, propylene-ethylene copolymer waxes like Clariant's Licocene PP-1502, oxidized polyethylene like Honeywell's A-C 6702 and A-C 330 and Henkel's Technomelt line of polyolefins. Polyamides like Henkel's Macromelt 6072. Other polymers for hot-melt compositions that can function as primary binding agents in the present invention include acrylic polymers and copolymers between olefins and acrylic monomers like for example polymethyl methacrylate, ethylene-acrylic acid copolymers (EAA) like Honeywell's A-C 5120, fully and partially neutralized salts of the ethylene-acrylic acid copolymers, ethylene-ethyl acetate, polyacrylates. Other suitable polymers for the present wetness indicator hot-melt compositions include oxidized ethylene-vinyl acetate copolymers like Honeywell's A-C 645P, ethylene maleic anhydride copolymers, propylene maleic anhydride copolymers, polyethylene imines (PEI) like BASF's Lupasol, polyurethanes like the polycaprolactone thermoplastic polyurethane named Pearlbond™ 120 from Lubrizol Inc., polyacryl amides, branched copolymers comprising monomeric units derived from acrylic acid and/or quaternary ammonium compounds and/or acrylamide, branched copolymers comprising one or more monomeric units derived from quaternary ammonium compounds, amine compounds, acrylamide compounds, acrylic acid compounds and mixtures thereof at various weight ratios within the polymer.

Other polymers that can make up the hot-melt matrix of the present compositions include polyamines, polypyrroles, polyimidazoles, polycarbonates, polyesters, styrene block copolymers, PVP, PVP/VA copolymers like Ashland Chemical's S-630 PVP/VA, polyacrylamide, polyacryldextran, polyalkyl cyanoacrylate, cellulose acetate, cellulose acetate butyrate, cellulose nitrate, methyl cellulose and other cellulose derivatives, chitosan and chitosan derivatives, chitin and chitin derivatives, nylons and other polyamides, polycaprolactones, polydimethylsiloxanes and other siloxanes, aliphatic and aromatic polyesters, polyethylene oxide, polyglycols, polyglycolic acid, polylactic acid and copolymers, poly(methyl vinyl ether/maleic anhydride), polystyrene, polyvinyl acetate phthalate, polyvinyl alcohol and its copolymers, shellac, starch and modified starches, fatty alcohols, primary alcohols of long carbon chain lengths of C14 to C60, ethoxylated fatty alcohols, ethoxylated primary alcohols having chain lengths of C14 to C60, fatty acids, and waxes such as paraffinic and microcrystalline, synthetic waxes like polyethylene waxes, natural waxes like beeswax, carnauba wax and mixtures thereof. As noted previously, some of these waxes and higher molecular weight materials can function also as hardeners.

The polymeric binding agent or agents may be employed in compositions at levels which are effective at immobilizing and stabilizing the pH-indicator colorant in its first state, including from about 1% to about 90%, from about 10% to about 75%, and from about 15% to about 65%, by weight of the wetness indicator hot-melt composition.

Additional components of the hot-melt binding matrix may include in addition to polymers, also tackifiers, waxes, plasticizers, wetting agents/surfactants, and/or anti-oxidants.

Tackifiers suitable for the hot-melt matrix include, without being limited to, natural resins like the copal type, the damar type, the mastic type, the sandarac type, and mixtures thereof; rosins, their esters and their modified derivatives, both fully and partially hydrogenated, like modified tall oil rosins with Sylvaros PR-R from Arizona Chemical being an example; polymerized rosins like Sylvaros PR 295 from Arizona Chemical™, partially dimerized gum rosins like Eastman Chemical Inc.'s Poly-Pale™, terpenes and modified terpenes; aliphatic, cycloaliphatic, and aromatic resins like C5 aliphatic resins, C9 aromatic resins, and C5/C9 aromatic/aliphatic resins, acidic rosins and acidic hydrogenated resins like Pinova's Foral AX synthetic resin, Eastman Chemical's fully hydrogenated rosin like its Foral AX-E, alkyl resins, phenolic resins and terpene-phenolic resins like Sylvare TP-2040 from Arizona Chemical Inc., hydrogenated hydrocarbon resins and their mixtures.

Tackifiers may be employed in the present wetness indicator hot-melt compositions at levels from about zero to about 60% or from about zero to about 40%, by weight of the composition.

Waxes suitable for the hot-melt matrix include, without being limited to, synthetic waxes like paraffin and microcrystalline waxes; polyethylene waxes; polyethylene glycol and polypropylene glycol type waxes like those trademarked as the Carbowax brand; oxidized polyethylene waxes; polymethylene waxes, the bis-stearamides like N,N′-ethylene bis-stearamide trademarked as Acrawax from Lonza Incorporated, highly branched polymer waxes like Vybar from Baker Hughes; fatty amide waxes; waxes that are copolymers of ethylene or propylene with maleic anhydride and/or maleic esters; natural and synthetic waxes like beeswax, soywax, carnuba, ozokerite, ceresin, montan wax; waxes derived from both the Fisher-Tropsch and Ziegler-Natta processes; water soluble waxes, polyalkylene wax, and silicone waxes. Many of these waxes are especially suitable for hot-melt type wetness indicators due to their ability to function as hardening agents.

Waxes may be employed in the wetness indicator compositions at levels from about zero to about 80% or from about zero to about 70%, by weight of the composition.

Additional components for the hot-melt matrix may include plasticizers, like glyceryl tribenzoate, benzoate esters like Eastman™ Chemicals Benzoflex™ 9-88, alkyl benzoates, C12-15 alkyl benzoate like Akzo's Dermol 25B, C2-C22 alkyl benzoates where the alkyl group is straight or branched or mixtures thereof, alkyl citrates, phthalate esters, paraffin oils, silicone oils, and polyisobutylene; UV stabilizers; biocides and antimicrobial preservatives; antioxidants, like BHT, phospites and phosphates; antistatic agents; pigment, particle and powder wetting agents like polyhydroxystearic acid, polyglyceryl-4 isostearate, hexyl laurate, esters like isopropyl myristate, propylene carbonate, isononyl isononanoate, glyceryl behenate/eicosadioate, trihydroxystearin, C12-15 alkyl benzoate, castor oil; and viscosity modifiers.

The hotmelt matrix may contain also mineral fillers, provided that they do not interfere with the color change of the pH indicators contained in the composition of the present invention. For example, an acidic filler like precipitated silica may function both as an effective hardener of the formulation while keeping the desired acidic environment.

The matrix, including both the first and second binding agents, may be employed in the present wetness indicator hot-melt compositions at levels which are effective at immobilizing and stabilizing the pH-indicator colorant, including from about 5% to about 95%, from about 10% to about 80%, and from about 25% to about 75%, by weight of the wetness indicator hot-melt composition.

### Additional Ingredients

Additional ingredients may include, for example, at least a wetting agent/surfactant or a blend of surfactants

Surfactants that are suitable for the present invention may be surfactants belonging to various chemical classes like anionic, cationic, zwitterionic and non-ionic surfactants. In one embodiment, preferred surfactants used in the wetness indicator hot-melt compositions of the present invention, are non-ionic surfactants.

Examples of suitable surfactants may include, for example, ethoxylated alcohols, fatty alcohols, high molecular weight alcohols, sorbitan esters, ethoxylated sorbitan esters like Tween 40 from Croda, the ethoxylated pareth surfactants like Performathox 420 and Performathox 450 and Performathox 480 and mixtures thereof from Baker Hughes Inc. Inc., ethoxylated esters, glycerol based esters, derivatized polymers; anionic and cationic and amphoteric surfactants, alkoxylated alkylates such as PEG-20 stearate, ethoxylated alcohols like the BRIJ materials from Croda Incorporated where Brij S-20/Steareth-20 and Brij L-23 and Brij S2/Steareth-2 are examples, end group-capped alkoxylated alcohols, alkoxylated glyceryl and polyglyceryl alkylates such as PEG-30 glyceryl stearate, glyceryl alkylates such as glyceryl stearate, low HLB emulsifiers like sorbitan esters where Span 60 from Croda Inc. is an example, alkoxylated hydrogenated castor oil, alkoxylated lanolin and hydrogenated lanolin, alkoxylated sorbitan alkylates, sugar derived surfactants such as the alkyl glycosides and sugar esters, poloxamers, polysorbates, and sulfo succinic acid alkyl esters like Aerosol OT-SE from Cytec is an example. Further examples include non-ionic surfactants and amphoteric surfactants and any combination thereof; specifically diethylhexyl sodium sulfosuccinate, available as MONOWET MOE75 from Croda, the sodium dioctyl sulfosuccinate line of surfactants like Aerosol OT-100 from Cytec Inc., the phosphate ester surfactants like Croda's Cetyl Phosphate tradenamed as Crodafos MCA or Croda's potassium salt form of Cetyl Phosphate tradenamed as Arlatone MAP160K, or Clariant's Cetyl Phosphate tradenamed as Hostaphat CC-100 and mixtures thereof, the alkyl benzene sulfonic acid and alkyl sulfonic acid surfactants and their corresponding salts like dodecylbenzene sulfonic acid tradenamed by AkzoNobel as Witconic 1298 Soft Acid or the counterpart with branching in the alkyl chain and tradenamed by AkzoNobel as Witconic 1298 Hard Acid, and mixtures thereof. Another example is 4-1-aminoethylphenolpolyoxyethylene fatty ethers, polyoxyethylene sorbitan esters, and polyoxyethylene fatty acid esters.

Other suitable surfactants may be neutral block copolymer surfactants, which can be selected from polyoxypropylene-polyoxyethylene block copolymer, poly [poly(ethylene oxide)-block-poly(propylene oxide)]copolymer or propylene glycol-ethylene glycol block copolymer. Suitable neutral polymeric surfactants include TWEEN surfactants, such as TWEEN 20 surfactant, TWEEN 40 surfactant and TWEEN 80 surfactant, and TRITON X-100 surfactant, which are available from Sigma-Aldrich, Incorporated. Other suitable neutral surfactants include polyethylene lauryl ether, polyoxyethylene nonyl phenyl ether, polyoxyethylene oleyl phenyl ether, polyoxyethylene sorbitan monolaurate, polyethylene glycol monostearate, polyethylene glycol sorbitan monolaurate, polyoxyethylenesorbitan monopalmitate, polyoxyethylenesorbitan monostearate, polyoxyethylenesorbitan monooleate, polyoxyethylenesorbitan trioleate, polypropylene glycol sorbitan monolaurate, polyoxypropylenesorbitan monopalmitate, polyoxypropylenesorbitan monostearate, polyoxypropylenesorbitan monooleate, polyoxypropylenesorbitan trioleate, polyalkyne glycol sorbitan monolaurate, polyalkyne glycol sorbitan monopalmitate, polyalkyne glycol sorbitan monostearate, polyalkyne glycol sorbitan monooleate, polyalkyne glycol sorbitan trioleate and mixtures of such neutral surfactants.

The neutral block copolymer based surfactants include PLURONIC series block copolymers, such as PLURONIC P84 or PLURONIC P85 surfactants, which are available from BASF Corporation.

Other suitable neutral block copolymer based surfactants include nonylphenol ethoxylates, linear alkyl alcohol ethoxylate, ethylene oxide-propylene oxide block copolymer, polyoxypropylene-polyoxyethylene block copolymer, polyalkylene oxide block copolymer and propylene glycol-ethylene glycol block copolymer.

When present, such surfactant or blend of surfactants are typically employed in the present hot-melt composition at levels that are effective at providing the benefits of the ingredient or ingredients, such as, for example, from about 0.001% to about 50%, from about 0.1% to about 40%, or from about 1% to about 35%, by weight of the composition.

It may be desirable to include additional stabilizer(s) of different types, when the absorbent article could be stored under conditions of high humidity and high temperature or ultra-intense UV light conditions. The inclusion of a color-stabilizer and a UV light absorber or both is also especially important for new absorbent article designs where materials and/or chemicals are present that could potentially prematurely activate the color change of the pH-indicator colorant within the formulation. Also, the wetness indicator composition may be heated and mixed for long times and at high temperatures, where the inclusion of anti-oxidants can slow down the degradation process. Thus, anti-oxidants like Irganox 1010 from BASF Inc. or Alvinox 100 from 3V-Sigma Inc. can aid in preventing premature oxidation and degradation of ingredients within the wetness indicator composition. In addition, if the wetness indicator composition might be exposed to ultraviolet light or intense sunlight for long periods of time, a UV stabilizer like Uvasorb S130 from 3V-Sigma or Escalol 577 (benzophenone-4, CAS #6628-37-1) from Ashland Chemicals might be added to inhibit photo-bleaching of the wetness indicator composition. Other effective UV stabilizers from BASF include Tinuvin-928 and Tinuvin-770 and Tinuvin-(384-2) and Tinuvin-123 and mixtures thereof.

Desiccants can stabilize the composition by trapping free water that could prematurely activate/pre-trigger the wetness indicator hot-melt composition. Examples of suitable desiccants include silica gel, bentonite clays, activated alumina, anhydrous calcium sulphate, copper(II) sulphate, and magnesium sulphate.

### Rheology Measurements Details

The rheometer used for the rheology measurements was a TA Instruments AR-G2 stress-controlled rheometer equipped with a TA Instruments ETC oven attachment.

The following program settings were used for the Oscillatory Temperature Ramp sequence for acquisition of G′ (pascals) and Temperature (degrees Celsius) values, in measuring the value of the Hot to Cold Solidification Rate expressed in Pa/(° C.).

### Instrument Type: TA-Instruments Model ARG2

Start temperature: 145° C.
Soak time: 300.0 seconds
Cooling rate: 2° C./min
End temperature: 5° C.

### Strain %: 0.01%

Sampling interval 30 seconds per data point

### Frequency: 0.5 Hz

1. A wetness indicator hot-melt composition, outside of the field of articles for baby and feminine care, having a melt point temperature from about 58° C. to about 135° C. and comprising:
a) at least one pH-indicator colorant
b) from about 0.001% to about 75% by weight of at least one color-stabilizer
c) from about 0.1% to about 70% by weight of at least one hardening agent said wetness indicator hot-melt composition having a hot to cold solidification rate Delta (G′)/Delta (° C.) from about 3,800 to about 27,000 Pa/° C.

2. The wetness indicator hot-melt composition of claim 1, where said composition has a hardness, expressed as a Needle Penetration measured according to ASTM D 1321-04, that is no greater than about 40 dmm at 23° C. and no greater than about 170 dmm at 55° C.

3. The wetness indicator hot-melt composition of claim 1, wherein the hardening agent comprises a long-chain alkyl chain moiety having a number of carbon atoms comprised between C12 and C70.

4. The wetness indicator composition of claim 1, wherein the hardening agent is acidic.

5. The wetness indicator hot-melt composition of claim 1, wherein the hardening agent has a number of carbon atoms comprised between C12 and C70 and is selected from the group consisting of linear primary carboxylic acids, polyolefin waxes, paraffin waxes, oxidized polyolefin waxes, maleic anhydride modified waxes, montan waxes and esters, C12-C50 fatty alcohols, C12-C50 fatty acids, hydrogenated vegetable oils, semi-crystalline polymers, polyesters, sorbitan esters and other high molecular weight esters, sucrose esters, and combinations thereof

6. The wetness indicator hot-melt composition of claim 1, wherein the hardening agent is a long chain, linear primary carboxylic acid having a number of carbon atoms comprised between C12 and C70.

7. The wetness indicator hot-melt composition of claim 1, wherein said composition comprises at least about 10% by weight of a hardening agent.

8. The wetness indicator hot-melt composition of claim 1, wherein said composition comprises from about 5% to about 60% by weight of a hardening agent.

9. The wetness indicator hot-melt composition of claim 1, wherein said composition comprises at least about 50% by weight of the hardening agent.

10. The wetness indicator hot-melt composition of claim 1, wherein the at least one pH-indicator colorant is selected from the group consisting of the free acid of bromophenol blue, the free acid of bromocresol green, the free acid of bromocresol purple and combinations thereof.

11. The wetness indicator hot-melt composition of claim 1, wherein the hot-melt binding matrix comprises one or more components selected from the group consisting of a thermoplastic polymer, a tackifier, a surfactant, an anti oxidant, UV stabilizers, plasticizers, waxes and combinations thereof

12. The wetness indicator hot-melt composition of claim 11, wherein the hot-melt matrix comprises at least one component selected from the group consisting of thermoplastic acidic copolymers, acidic tackifiers, acidic waxes, acidic plasticizers and combinations thereof.

13. The wetness indicator hot-melt composition of claim 10, wherein the surfactant or surfactants are non-ionic.

14. The wetness indicator hot-melt composition of claim 1, wherein the color-stabilizer is selected from the group consisting of linear primary carboxylic acids, acidic waxes, acidic phosphate esters, acidic rosin esters, copolymers of ethylene with acrylic or methacrylic acid or combinations thereof.

15. The wetness indicator hot-melt composition of claim 1, wherein said composition has a hot to cold solidification rate of Delta(G′)/Delta(° C.) from about 4,800 to about 22,600 Pa/° C.

16. The wetness indicator hot-melt composition of claim 1, wherein said composition has a hot to cold solidification rate of Delta(G′)/Delta(° C.) from about 5,800 to about 17,800 Pa/° C.

17. An article, outside of the field of articles for baby and feminine care, wherein said article comprises a wetness indicator hot-melt composition that comprises:
a) at least one pH-indicator colorant
b) from about 0.001% to about 75% by weight of at least one color-stabilizer
c) from about 0.1% to about 70% by weight of at least one hardening agent said wetness indicator composition having a hardness, expressed as a Needle Penetration measured according to ASTM D 1321-04, that is no greater than about 40 dmm at 23° C. and no greater than about 170 dmm at 55° C.

18. An article comprising the wetness indicator hot-melt composition of claim 1, outside of the field of articles for baby and feminine care, wherein said wetness indicator hot-melt composition is applied on a structural component of the article, said structural component being such that it can be used as an adhesive element of the article.

19. The article of claim 18, wherein said article is an absorbent mat or sheet or a wound dressing product for medical use.

20. The article of claim 18, wherein said article is an absorbent mat or sheet for keeping dry food products.
